(19) United States
(12) Patent Application Publication
(10) Pub. No.: US 2020/0279640 A1
(43) Pub. Date: Sep. 3, 2020

(54) AUTOMATED ASSISTANCE TO STAFF AND QUALITY ASSURANCE BASED ON REAL-TIME WORKFLOW ANALYSIS

(71) Applicant: KONINKLIJKE PHILIPS N.V., EINDHOVEN (NL)

(72) Inventors: Thomas Erik AMTHOR, HAMBURG (DE); Tanja NORDHOFF, HAMBURG (DE); Joachim SCHMIDT, HAMBURG (DE); Joern BORGERT, HAMBURG (DE); Ingmar GRAESSLIN, BOENNINGSTEDT (DE)

(73) Assignee: KONINKLIJKE PHILIPS N.V., EINDHOVEN (NL)

(21) Appl. No.: 16/649,698

(22) PCT Filed: Sep. 25, 2018
(86) PCT No.: PCT/EP2018/076018

§ 371 Date: Mar. 23, 2020

Related U.S. Application Data
(60) Provisional application No. 62/563,127, filed on Sep. 26, 2017.

(51) Int. Cl. G16H 40/20; G16H 30/20; G16H 40/67; G16H 70/20; G06Q 10/06; A61B 5/055; A61B 5/00; A61B 6/03; A61B 6/00; A61B 8/00

(52) U.S. Cl. CPC G16H 40/20; G16H 30/20; G16H 40/67; G16H 70/20; G06Q 10/06316; A61B 8/54; A61B 5/0046; A61B 6/032; A61B 6/037; A61B 6/54; A61B 5/055

(57) ABSTRACT

An imaging system (10) includes: an image acquisition device (12), a device controller (16) comprising an electronic processor (20) programmed to operate the image acquisition device to acquire medical images of a patient and to maintain a machine log (26) storing an operating history of the image acquisition device; a server computer (34) programmed to retrieve patient information from at least one health information system (HIS) (18); and at least one feedback device (50, 52). The device controller, the server computer, or a combination of the device controller and server computer is programmed to implement at least one state machine (30, 44) having a plurality of states defined by values of state variables wherein the states represent respective attainable states of an image acquisition procedure (100) and the image acquisition device. The at least one state machine is configured to transition between states during the image acquisition procedure whereby the at least one state machine represents a current state of the image acquisition procedure and the image acquisition device, wherein the values of the state variables of the at least one state machine are determined based at least on content of the machine log for the image acquisition device and patient information retrieved by the server computer from the at least one HIS. The at least one feedback device is configured to provide guidance to an operator for performing the image acquisition procedure based on a current state of the at least one state machine.

### FIELD

The following relates to the medical arts, clinical decision support arts, automated medical data analytics arts, and related arts.

### BACKGROUND

A difficulty with expansion of the use of complex medical imaging systems is the need for trained operators. Currently, developing countries (e.g., Brazil) may not have access to complex imaging systems, such as Magnetic Resonance (MR), computed tomography (CT), positron emission tomography (PET), single photon emission computed tomography (SPECT), ultrasound (US), and the like. Even if such countries have these systems, medical personnel in these countries may not have proper training or skills to operate these devices. While the problem is less severe in developed countries, wait times for a medical imaging session can still be lengthy, and this can be especially problematic in time-critical medical situations such as a rapidly spreading cancer or a person suffering from a broken (or suspected broken) bone. In many medical imaging laboratories, limited personnel availability results in medical imaging devices being used only during normal (“day shift”) working hours, introducing an inherent inefficiency as valuable medical imaging devices are not utilized during off-hours (e.g., during the “night shift”).

In the case of developing countries, remote command centers may be set up in these countries to oversee or supervise and train medical personnel to use these devices. However, these command centers can be expensive, time-consuming, and may not be staffed well.

Another difficulty with existing medical imaging procedures relates to documentation of executed imaging procedures. These imaging procedures should be documented for use in future operations, and/or to establish an auditable record of imaging procedures, for training purposes, and/or so forth. Currently, machine logs are maintained in many cases, but these logs are generally at a low level and may not be easily related to specific imaging sessions.

The following discloses new and improved systems and methods to overcome these problems.

### SUMMARY

In one disclosed aspect, an imaging system includes: an image acquisition device; a device controller comprising an electronic processor programmed to operate the image acquisition device to acquire medical images of a patient and to maintain a machine log storing an operating history of the image acquisition device; a server computer programmed to retrieve patient information from at least one health information system (HIS); and at least one feedback device. The device controller, the server computer, or a combination of the device controller and server computer is programmed to implement at least one state machine having a plurality of states defined by values of state variables wherein the states represent respective attainable states of an image acquisition procedure and the image acquisition device. The at least one state machine is configured to transition between states during the image acquisition procedure whereby the at least one state machine represents a current state of the image acquisition procedure and the image acquisition device, wherein the values of the state variables of the at least one state machine are determined based at least on content of the machine log for the image acquisition device and patient information retrieved by the server computer from the at least one HIS. The at least one feedback device is configured to provide guidance to an operator for performing the image acquisition procedure based on a current state of the at least one state machine.

In another disclosed aspect, a non-transitory computer-readable medium stores instructions readable and executable by a computer including at least one electronic processor to perform an image acquisition method. The method includes: controlling an image acquisition device to acquire one or more images of a patient; updating a machine log of the image acquisition device during the acquiring of the images; retrieving information from at least one of a health information system (HIS), a first database storing information about typical imaging workflow patterns from previous image acquisitions procedures, and a second database including information about imaging site-specific standardized workflows information about the patient being scanned; implementing a current state of at least one state machine of the computer during acquisition of the images based on the retrieved information and the updated machine log; controlling at least one feedback device operatively connected with the image acquisition device and the computer to provide guidance to an operator of the image acquisition device based on the current state of at least one of the state machines; and updating at least one of the state machines as the at least one state machine transitions between states based on the received guidance.

In another disclosed aspect, an imaging system includes an image acquisition device. A device controller includes an electronic processor programmed to operate the image acquisition device to acquire medical images of a patient and to maintain a machine log storing an operating history of the image acquisition device. The device controller is programmed to implement a first state machine having a plurality of states defined by values of state variables wherein the states represent respective attainable states of the image acquisition device. The first state machine is configured to transition between states during an image acquisition procedure whereby the first state machine represents a current state of the image acquisition device. The values of the state variables of the first state machine are determined based at least on content of the machine log. The state variables of the first state machine include at least a patient position and settings of the image acquisition device. A server computer is programmed to retrieve patient information from at least one health information system (HIS). The server computer is programmed to implement a second state machine having a plurality of states defined by values of state variables wherein the states represent respective attainable states of the image acquisition procedure. The second state machine is configured to transition between states during the image acquisition procedure whereby second state machine represents a current state of the image acquisition procedure. The values of the state variables of the second state machine are determined based at least on content of the patient information retrieved by the server computer from the at least one HIS. The state variables of the second state machine include at least an identification of the image acquisition procedure and patient medical information retrieved from the at least one HIS. At least one database is operatively connected with the server computer. The at least one database stores information related to image acquisition workflow patterns from previous image acquisition procedures, information about standardized workflows of the image acquisition device and information about the patient being scanned during the image acquisition procedure. The second state machine is updated with information from the at least one database. At least one feedback device is configured to provide guidance to an operator for performing the image acquisition procedure based on a current state of the first and second state machines.

One advantage resides in providing a system to automatically provide timely information to an imaging device operator at the right time, thereby minimizing the chance for errors, minimizing the need for involving another person, and increasing efficiency.

Another advantage resides in providing a method to document quality-related information during an imaging examination by automatically asking the operator the right, relevant, or important questions at the appropriate time.

Another advantage resides in providing a system to provide additional information to detect a mismatch between an imaging examination order and the actual imaging procedure to be carried out.

Another advantage resides in more efficient integrated use of quality assurance tools.

Another advantage resides in reduced manual data entry in using quality assurance tools.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an imaging system according to one aspect; and

FIG. 2 shows an exemplary flow chart operation of the imaging system of FIG. 1.

### DETAILED DESCRIPTION

The following relates to a system for providing guidance to the medical imaging device operator during a medical imaging examination and creating a quality assurance (QA) record of the medical imaging examination in furtherance of operator training, data mining to improve workflow, post-examination review, and/or so forth.

The illustrative system represents a magnetic resonance imaging (MRI) examination workflow as a state machine. This flexible representation allows for representing the extensive run-to-run variability by providing states and state transitions to represent various pathways that may be followed in any MRI examination instance. For example, states and state transitions can be created to represent events such as patient motion during a scan (e.g., a transition that returns to the same state to repeat the scan), different orders of performing scans, and so forth. Each state is defined by a certain space of values for the state variables (which may store patient parameters, imaging device parameters, examination progress information, or so forth), and each state may have a range of actions that are performed when that state is reached (e.g., displaying instructional video, providing recommendations to the operator, or so forth).

To capture the quality assurance recordation aspect, each state also may have feedback fields that are filled in when the examination enters the state. These may be filled in by way of user input received via user graphical user interface (GUI) dialogs, or by reading automatically generated machine log entries, or by reading patient vital sign sensors, and/or so forth. In one contemplated aspect, the state machine is designed to implement the expected work flow, and if a particular MRI examination deviates from that flow then the operator may be asked to provide feedback as to the reason for the deviation. Such feedback may be solicited, for example, if the operator does not follow a recommendation provided by the system, or if a task takes longer than expected, or if an unusual event occurs, and/or so forth.

The system may be implemented on the scanner host computer and/or on a remote application server. A GUI may be provided via which an expert MRI technologist drafts the state diagram of the state machine including defining the states and various state transitions. Thereafter, in a machine learning phase this state machine is applied to historical MRI examination data. The machine learning provides for determining probabilities of the various state transitions (thus capturing probabilities of various events occurring as events correspond to state transitions), and also provides an efficient mechanism for detecting situations that are not represented by the current state diagram (the expert MRI technologist can then update the state diagram to account for these situations).

After creating the state machine, it is used to provide operator assistance and QA recordation during MRI examinations. The operator experiences the system by way of receiving recommendations, e.g. for customizing the exam card, performing various operations during the examination, or so forth) and being asked to provide feedback at certain points. In execution, the system receives input data about the patient from the Health Information System (HIS) or other medical database(s), and input data about the medical imaging device state by reading the automatically generated machine logs, and input data about the intended examination (e.g. the clinical question being answered) from the MRI exam order in the Radiology Information System (RIS), and/or so forth.

MRI is described as an illustrative medical imaging device to which the disclosed improvements may be usefully applied. However, the disclosed systems and methods are not limited to MRI but rather extends to medical imaging devices generally, e.g. MRI, CT, PET, SPECT, et cetera.

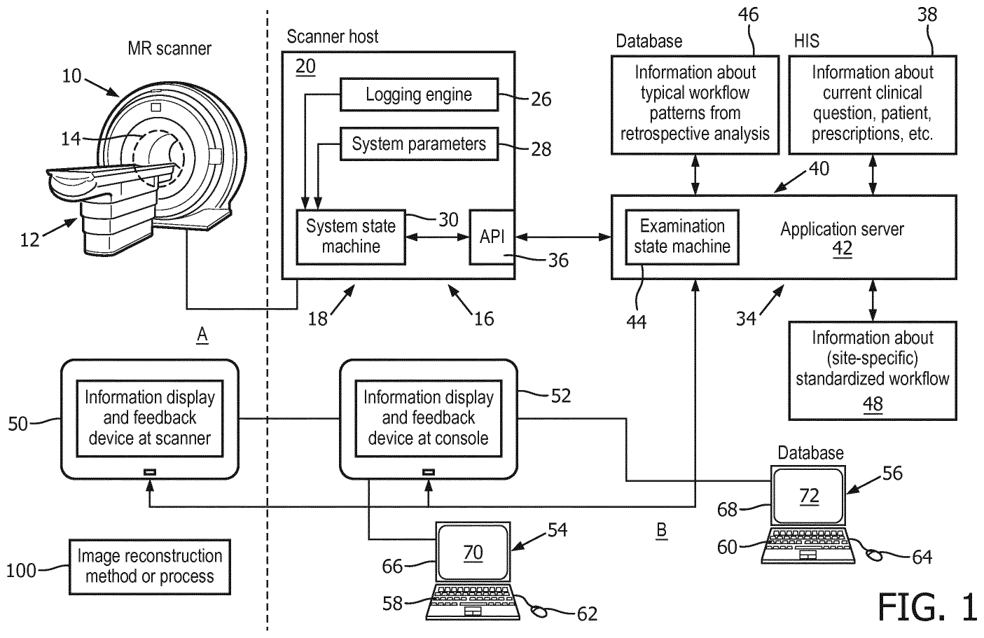

With reference to FIG. 1, an illustrative medical imaging system 10 is shown. As shown in FIG. 1, the system 10 includes an image acquisition device 12. In one example, the image acquisition device 12 can comprise a Magnetic Resonance (MR) imaging device arranged to collect imaging data from a patient disposed in an examination region 14. However, it will be appreciated that the image acquisition device 12 can include any suitable imaging device (e.g., a computed tomography (CT) device, a positron emission tomography (PET) device, a gamma camera for a single photon emission computed tomography (SPECT) device, and an ultrasound (US) device). The image acquisition device 12 is configured to obtain images of a patient during an image acquisition procedure 100 using known MRI, CT, PET, SPECT, US, et cetera imaging data acquisition and image reconstruction methodologies.

The imaging system 10 also includes a scanner host or device controller 16 operatively connected with the MR scanner 12. The scanner host 16 can be implemented as a computer or workstation or other electronic data processing device 18 with typical components, such as at least one electronic processor 20. The device controller 16 also includes a machine logging engine or machine log 26 which stores an operating history (e.g., number of scans, patients scanned, accessory installation/removal events, and the like) of the MR scanner 12. While a single machine log 26 is shown, it will be appreciated that the machine logs may be organized into two or more logs, e.g. a machine log storing low level machine events, a service log storing servicing events, a laboratory log storing higher level information on patient imaging sessions, et cetera. The device controller 16 also includes a system parameters database 28 storing parameters of the MR scanner 12 (e.g., operating times, coil status, and the like). The at least one electronic processor 20 is programmed to operate the image acquisition device 12 to acquire medical images of a patient, and to maintain a machine log storing an operating history of the image acquisition device in the machine log 26.

The at least one electronic processor 20 is further programmed to implement a state machine 30 having a plurality of states defined by values of state variables. The states represent respective attainable states of the image acquisition device 12. The state machine 30 is configured to transition between states during operation of the MR scanner 12. The state machine 30 represents a current state of the image acquisition device 12. The values of the state variables of the state machine 30 are determined based at least on content of the machine log 26 for the image acquisition device 12. The state variables of the state machine 30 can include a patient position, settings of the MR scanner 12 retrieved from the system parameters database 28, patient vital signs retrieved from sensors (not shown) optionally attached to the patient for certain imaging procedures, patient motion during an imaging scan detected by such sensors or by other approaches such as detecting changes in images (e.g. image frames) acquired from one time interval to the next, different orders of performing scans, and so forth.

The imaging system 10 also includes a server computer 34 in communication with the device controller 16 to receive information from the device controller via an application programming interface (API) 36 disposed on the device controller. The server computer 34 is programmed to retrieve patient information from at least one health information system (HIS) 38 (e.g., an electronic health record (EHR), an electronic medical record (EMR), a cardiovascular information system (CVIS), a picture archiving and communication system (PACS), a radiology information system (RIS), and the like). The server computer 34 can be implemented as a computer or workstation or other electronic data processing device 40 with typical components, such as at least one electronic processor 42.

The server computer 34 is programmed to model a current state of the image acquisition procedure 100 based at least on content of the patient information retrieved by the server computer from the at least one HIS and data stored in the machine log 26. To do so, the at least one electronic processor 42 is programmed to implement an examination state machine 44 having a plurality of states by values of state variables. The states represent respective attainable states of the image acquisition procedure 100. The state machine 44 is configured to transition between states during the image acquisition procedure 100. The state machine 44 represents a current state of the image acquisition procedure 100. The values of the state variables of the state machine 44 are determined based at least on patient information retrieved by the server computer 34 from the at least one HIS 38 and/or the state of the state machine 30 of the device controller 16. The state variables of the state machine 44 include at least an identification of the image acquisition procedure 100 and patient medical information retrieved from the at least one HIS 38. In some examples, the retrieved patient medical information can include at least a state of patient motion during an imaging scan, a state of different orders of performing imaging scans, patient vital signs, a type of ongoing examination, an exact state of an ongoing image acquisition workflow, a probability for different actions to be performed next, scans that have been performed, scans that still need to be performed, actions still necessary to be performed, descriptions manuals, and guidelines for future steps in the image acquisition procedure 100, operations to follow and document to achieve quality assurance, and so forth.

In some embodiments, the server computer 34 is also in communication with one or more databases, including a first database 46 storing information about typical imaging workflow patterns from previous image acquisitions procedures, and a second database 48 including information about imaging site-specific standardized workflows information about the patient being scanned during the image acquisition procedure 100. The state machine 44 is updated from information from the first database 46 and/or the second database 48.

It will be appreciated that, in some embodiments, a first state machine (i.e., the state machine 30) and a second state machine (e.g., the state machine 44) can be implemented as a single state machine on either the device controller 16 or the server computer 34. The illustrative implementation as two separate state machines 30, 44, one of which is a system state machine 30 representing the state of time imaging device 12 and the other of which is an examination state machine 44 representing the state of the imaging examination, has certain advantages. For example, this division allows the system state machine 30 to be implemented on the scanner host 16 using information mostly or entirely local to the imaging laboratory, while the examination state machine 44 is implemented at the server computer 34 which has high bandwidth access to the various databases 38, 46, 48 and can leverage the typically greater computing capacity of the server computer 34 (which may, in some embodiments, comprise a network of servers, an ad hoc cloud computing resource, or so forth). The division into separate state machines 30, 44 also facilitates maintaining operational isolation of the scanner host 16 from the hospital-level computing network, which promotes cyber security for the scanner host 16.

The imaging system 10 also includes at least one feedback device. As shown in FIG. 1, the at least one feedback device includes a first feedback device 50 and a second feedback device 52. One or both of the feedback devices 50 and 52 can comprise a computer or workstation or other electronic data processing device 54, 56 with typical components, include at least one corresponding electronic processor 58, 60, at least one user input device (e.g., a mouse, a keyboard, a trackball, and/or the like) 62, 64, and a display device 66, 68 (such as an LCD display, projection display, or so forth). In some embodiments, the display devices 66, 68 can be separate components from the corresponding feedback devices 50, 52. In other embodiments, the display devices 66, 68 can be touch screens, and thus the user input devices 62, 64 constitute a finger movement (e.g., tapping or swiping) by the operator. Each of the display devices 66, 68 is configured to display a corresponding graphical user interface (GUI) 70, 72 including at least one GUI dialog (not shown) to receive inputs (e.g., from the user input devices 62, 64) from the operator.

The feedback devices 50 and 52 are configured to provide guidance to an operator for performing the image acquisition procedure 100 based on a current state of at least one of the state machines 30 and 44. For example, the states of the state machine 30 and/or the state machine 44 (or a single state machine) may be extant if one of the state variables is within a certain space. The states machines 30 and 44 each have associated actions to perform when one of the corresponding states is extant. The associated actions can include recommended actions, including one or more of displaying an instructional video to the operator, providing a recommendation (e.g., audio or visual) in the form of a message to the operator. The recommendations can include, for example, images or videos explaining how to position the patient for the specific examination to be performed; images or videos explaining how to administer a contrast agent when one of the state machines 30, 44 identifies an examination requiring the contrast agent and after all scheduled non-contrast agent enhanced scans are finished; information about the required contrast agent dose for the specific patient; advice about special assistance needed for a specific patient; proposals of what to say to the patient in a certain situation (e.g., when one of the state machines 30, 44 detects a repeated scan, so the patient should be calmed down to minimize motion); automatic translation of these proposals to a language spoken by the patient, preferably combined with a speech processing system in case the operator is not speaking the patient's language; proposals for a different scan type that is less sensitive to motion but still allows for the required diagnosis (when one of the state machines 30, 44 detects a repetition of a motion-sensitive scan), and so forth.

In some embodiments, these recommendations can be based on the information stored in one or more of the first and second databases 46, 48. The GUIs 70, 72 are configured to display the GUI dialogs to receive inputs from the operator indicative of the recommended actions (e.g., “accepted recommendation;” “reject recommendation”;

“request recommendation”; “perform scan;” “pause/resume scan” and so forth). In some examples, for each step in the image acquisition procedure 100, the operator can be asked to acknowledge that the procedure has been performed as proposed by the imaging system 10, or give a reason for a deviation from this proposal. When the operator does not follow the recommended actions, the GUIs 70, 72 can display a GUI dialog to receive a value via the inputs devices 62, 64 for a feedback field explaining why the recommended action was not followed. This feedback can be stored (e.g., in the one of the databases 46, 48) to establish a quality control system to document if certain quality related aspects or necessary steps/procedures have been followed by the operator during the examination. This guarantees a standardization of the process steps and increases reproducibility of the workflow, which also leads to an optimization of the workflow. Examples of feedback can include: an acknowledgement by the operator that the patient is positioned in exactly the way shown in an image on the display devices 66, 68 when a certain patient positioning should be used (e.g., for a shoulder or knee examination); an acknowledgement by the operator that the patient is correctly positioned in the image acquisition device 12 (e.g., the patient's left arm is in the imaging region, and not the right arm); a confirmation by the operator that a certain procedure has been explained to the patient; a request to the operator that extra checks are performed on the images according to a global/local quality assurance system; a request to the operator to initiate extra automated image quality checks (e.g. to detect if too much motion etc.), to be able to re-scan the patient immediately, opposed to calling him in at a later point in time, and so forth. When these feedbacks are combined with information from the databases 46, 48, the imaging system 10 can check or ask the operator to check that each element defined in the standardized workflow have been considered. Where feasible, such manually provided feedback can be cross-referenced with available machine log information and/or sensor data, e.g. if the operator asserts an operation has been performed but machine log entries are not consistent with this assertion, then follow-up feedback questions may be asked, and/or the discrepancy may be stored as part of the QA information.

The imaging system 10 can be configured in other ways. In one embodiment, to minimize an integral cost of the implementation (e.g., lower hardware cost, easier maintenance cost by using use of service access to the MR system, etc.) of the imaging system 10, the real-time analysis of the feedback devices 50, 52 is executed on the device controller 16, by retrieving additional required information via the API 36 to other components such as the databases 46, 48 databases and/or the HIS 38. The displays 66, 68 of the feedback devices 50, 52 are connected to the device controller 16 or by the server computer 34. In another example, the first feedback device 50 is built directly into the image acquisition device 12.

In another embodiment, the server computer 34 is also connected to a remote command center (not shown). Information about the state of the examination can thus be transferred automatically to a remote consultant. When a communication channel to the remote command center is opened on request of the operator, the necessary information about the examination state is already available. In another example, the feedback devices 50, 52 serve as communication devices with a remote consultant (not shown), e.g. by providing an audio or video connection. In another embodiment, the quality-control-related information from the feedback devices 50, 52 is transferred into a hospital IT system (not shown) to allow for hospital-wide access, documentation, and archiving either by means of unsolicited messages or on request (query).

In a more specific embodiment, as further shown in FIG. 1, the imaging system 10 includes the image acquisition device 12, the device controller 16, the server computer 34, the databases 46 and 48 and the first and second feedback devices 50 and 52. In some examples (e.g., when the image acquisition device 12 is an MR scanner), the MR scanner, and the first feedback device 50 can be disposed in a first room A with a radio-frequency (RF) shield (indicated by the dashed line) in the walls of the first room. The device controller 16, server computer 34, the databases 46, 48, and the second feedback device 52 can be disposed in a separate second room B that does not include a RF shield. In another example, the device controller 16 and the server computer 34 may be disposed in separate, non-RF shielded rooms. Advantageously, the server computer 34, the databases 46, 48, and the second feedback device 52 are protected from RFs emitted by the MR scanner 12. In one embodiment, information about deviations from the standard workflow is used for adaptively changing and re-optimizing the room plan.

The at least one electronic processor 20 of the device controller 16 and/or the electronic processor 42 of the server computer 34 is/are operatively connected with a non-transitory storage medium (not shown) that stores instructions which are readable and executable by the at least one electronic processor(s) 20 (and/or 42) to perform disclosed operations including performing the image acquisition method or process 100. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth. In another embodiment, the image acquisition method or process 100 may be performed by cloud processing.

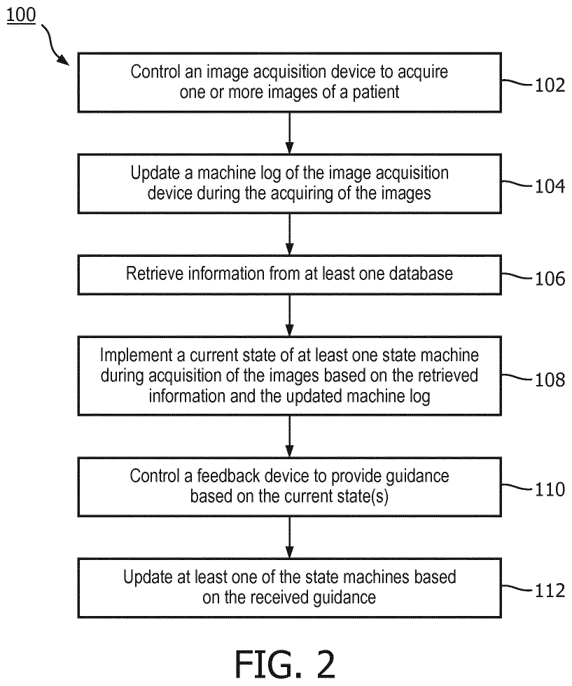

With reference to FIG. 2, an illustrative embodiment of the image acquisition method 100 is diagrammatically shown as a flowchart. At 102, the at least one electronic processor 20, 44 is programmed to control the image acquisition device 12 to acquire one or more images of a patient. At 104, the at least one electronic processor 20, 44 is programmed to update the machine log 26 during the acquiring of the images. At 106, the at least one electronic processor 20, 44 is programmed to retrieve information from at least one of the HIS 38, the first database 46, and the second database 48. At 108, the at least one electronic processor 20, 44 is programmed to implement a current state of at least one of the state machines 30, 44 during acquisition of the images based on the retrieved information and the updated machine log 26. At 110, the at least one electronic processor 20, 44 is programmed to control at least one of the feedback devices 50, 52 to provide guidance to an operator of the image acquisition device 12 based on the current state of at least one of the state machines 30, 44. At 112 the at least one electronic processor 20, 44 is programmed to update at least one of the state machines 30, 44 as the state machine(s) transition between states based on the received guidance.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An imaging system, comprising:
an image acquisition device;
a device controller comprising an electronic processor programmed to operate the image acquisition device to acquire medical images of a patient and to maintain a machine log storing an operating history of the image acquisition device;
a server computer programmed to retrieve patient information from at least one health information system (HIS); and
at least one feedback device;
wherein the device controller, the server computer, or a combination of the device controller and server computer is programmed to implement at least one state machine having a plurality of states defined by values of state variables wherein the states represent respective attainable states of an image acquisition procedure and the image acquisition device, the at least one state machine configured to transition between states during the image acquisition procedure whereby the at least one state machine represents a current state of the image acquisition procedure and the image acquisition device, wherein the values of the state variables of the at least one state machine are determined based at least on content of the machine log for the image acquisition device and patient information retrieved by the server computer from the at least one HIS; and
wherein the at least one feedback device is configured to provide guidance to an operator for performing the image acquisition procedure based on a current state of the at least one state machine.

2. The imaging system of claim 1, wherein the at least one state machine comprises:
a first state machine representing the imaging acquisition device and having a plurality of states, wherein the values of the state variables of the first state machine are determined based on content of the machine log for the image acquisition device; and
a second state machine representing the image acquisition procedure and having a plurality of states, wherein the values of the state variables of the second state machine are determined based at least on the first state machine and patient information retrieved by the server from the at least one HIS.

3. The imaging system of claim 2, wherein:
the state variables of the first state machine include at least a patient position and settings of the image acquisition device, and
the state variables of the second state machine include at least an identification of the image acquisition procedure and patient medical information retrieved from the at least one HIS.

4. The imaging system of claim 1, wherein the at least one state machine has a plurality of states including at least a state of patient motion during an imaging scan and a state of different orders of performing imaging scans.

5. The imaging system of claim 1, wherein the states of the at least one state machine have associated actions to be performed when one of the corresponding states is extant.

6. The imaging system of claim 5, wherein the associated actions include at least displaying an instructional video to the operator and providing recommendations to the operator.

7. The imaging system of claim 5, wherein the associated actions include recommended actions and, conditional upon the operator not following a recommended action,
wherein the at least one feedback device is configured to display a graphical user interface (GUI) including at least one GUI dialog configured to receive a value for a feedback field explaining why the recommended action was not followed.

8. The imaging system of claim 3, wherein the state variables further include patient vital sign values.

9. The imaging system of claim 6, wherein the at least one feedback device is configured to display a graphical user interface (GUI) including at least one GUI dialog configured to receive an input by the operator indicative of at least one of the recommended actions.

10. The imaging system of claim 2, further including at least one database operatively connected with the server computer, the at least one database storing information related to image acquisition workflow patterns from previous image acquisition procedures, information about standardized workflows of the image acquisition device, and information about the patient being scanned during the image acquisition procedure,
wherein the second state machine is updated with information from the at least one database.

11. The imaging system of claim 10, wherein the at least one feedback device is configured to provide recommendations to the operator during the image acquisition procedure by at least one of audio or visual message, the recommendations being based on the information in the at least one database.

12. The imaging system of claim 1, wherein the image acquisition device is one of a magnetic resonance imaging (MRI) device, a computed tomography (CT) device, a positron emission tomography (PET) device, a single photon emission computed tomography (SPECT) device, or an ultrasound (US) device;
wherein the at least one feedback device includes a first feedback device and a second feedback device, and
wherein the first feedback device, and the image acquisition device are located in a first, radio-frequency (RF) shielded room, and the device controller, the second feedback device and the image acquisition application server are located in a second separate, non-RF shielded room.

13. A non-transitory computer-readable medium storing instructions readable and executable by a computer including at least one electronic processor to perform an image acquisition method, the method comprising:
controlling an image acquisition device to acquire one or more images of a patient;
updating a machine log of the image acquisition device during the acquiring of the images;
retrieving information from at least one of a health information system (HIS), a first database storing information about typical imaging workflow patterns from previous image acquisitions procedures, and a second database including information about imaging site-specific standardized workflows information about the patient being scanned;
implementing a current state of at least one state machine of the computer during acquisition of the images based on the retrieved information and the updated machine log;
controlling at least one feedback device operatively connected with the image acquisition device and the computer to provide guidance to an operator of the image acquisition device based on the current state of at least one of the state machines; and
updating at least one of the state machines as the at least one state machine transitions between states based on the received guidance.

14. The non-transitory computer-readable medium of claim 13, wherein the at least one state machine comprises:
a first state machine representing the imaging acquisition device and having a plurality of states, wherein the values of the state variables of the first state machine are determined based on content of the machine log for the image acquisition device; and
a second state machine representing the image acquisition procedure and having a plurality of states, wherein the values of the state variables of the second state machine are determined based at least on the first state machine and patient information retrieved by the server from the at least one HIS.

15. The non-transitory computer-readable medium of claim 14, wherein:
the state variables of the first state machine include at least a patient position and settings of the image acquisition device, and
the state variables of the second state machine include at least an identification of the image acquisition procedure and patient medical information retrieved from the at least one HIS.

16. The non-transitory computer-readable medium of claim 13, wherein the at least one state machine has a plurality of states including at least a state of patient motion during an imaging scan and a state of different orders of performing imaging scans; and
wherein the states of the at least one state machine have associated actions to be performed when one of the corresponding states is extant, the associated actions include at least displaying an instructional video to the operator, providing recommendations to the operator, and recommended actions.

17. The non-transitory computer-readable medium of claim 14, further including at least one database operatively connected with the computer, the at least one database storing information related to image acquisition workflow patterns from previous image acquisition procedures, information about standardized workflows of the image acquisition device, and information about the patient being scanned during the image acquisition procedure,
wherein the second state machine is updated with information from the at least one database; and
wherein the at least one feedback device is configured to provide recommendations to the operator during the image acquisition procedure by at least one of audio or visual message, the recommendations being based on the information in the at least one database.

18. An imaging system, comprising:
an image acquisition device;
a device controller comprising an electronic processor programmed to operate the image acquisition device to acquire medical images of a patient and to maintain a machine log storing an operating history of the image acquisition device;
a server computer operatively connected with the image acquisition device and programmed to retrieve patient information from at least one health information system (HIS), the server computer being programmed to model a current state of an image acquisition procedure based at least on content of the patient information retrieved by the server computer from the at least one HIS and data stored in the machine log;
at least one database operatively connected with the server computer, the at least one database storing information related to image acquisition workflow patterns from previous image acquisition procedures, information about standardized workflows of the image acquisition device and information about the patient being scanned during the image acquisition procedure, the second state machine being updated with information from the at least one database; and
at least one feedback device is configured to provide guidance to an operator for performing the image acquisition procedure based on the current state of the image acquisition procedure.

19. The imaging system of claim 18, wherein:
the device controller is programmed to implement first state machine having a plurality of states defined by values of state variables wherein the states represent respective attainable states of the image acquisition device, the first state machine configured to transition between states during the image acquisition procedure whereby the first state machine represents a current state of the image acquisition device, wherein the values of the state variables of the first state machine are determined based at least on content of the machine log, the state variables of the first state machine including at least a patient position and settings of the image acquisition device; and
the server computer is programmed to model the current state of the image acquisition procedure by implementing a second state machine having a plurality of states defined by values of state variables wherein the states represent respective attainable states of the image acquisition procedure, the second state machine configured to transition between states during the image acquisition procedure whereby second state machine represents a current state of the image acquisition procedure, wherein the values of the state variables of the second state machine are determined based at least on content of the patient information retrieved by the server computer from the at least one HIS, the state variables of the second state machine including at least an identification of the image acquisition procedure and patient medical information retrieved from the at least one HIS.

20. The imaging system of claim 19, wherein the server computer state machine and the image acquisition device state machine each have a plurality of states including at least a state of patient motion during an imaging scan and a state of different orders of performing imaging scans; and
wherein the states of the first and second state machines have associated actions to be performed when one of the corresponding states is extant, the associated actions include at least displaying an instructional video to the operator, providing recommendations to the operator, and recommended actions.
wherein the at least one feedback device is configured to provide recommendations to the operator during the image acquisition procedure by at least one of audio or visual message, the recommendations being based on the information in the at least one database.
